(19) United States
(12) Patent Application Publication
(10) Pub. No.: US 2011/0046120 A1
(43) Pub. Date: Feb. 24, 2011

(54) TREATMENT OF IMPULSE CONTROL DISORDERS

(75) Inventor: Igor Elman, Ashland, MA (US)

Correspondence Address: FOLEY & LARDNER LLP, P.O. BOX 80278, SAN DIEGO, CA, 92138-0278, US

(73) Assignee: Mclean Hospital Corporation

(21) Appl. No.: 12/446,512

(22) PCT Filed: Oct. 25, 2007
(86) PCT No.: PCT/US07/22708

§ 371 Date: Nov. 10, 2010

Related U.S. Application Data
(60) Provisional application No. 60/854,492, filed on Oct. 26, 2006.

(51) Int. Cl. A61K 31/54; A61K 31/138; A61K 31/165; A61K 31/404; A61K 31/166; A61K 31/167; A61K 31/5377; A61K 31/4704; A61K 31/403; A61K 31/24; A61K 31/18; A61K 31/353; A61K 31/433; A61K 31/155; A61K 31/195; A61K 31/4164; A61P 25/00

(52) U.S. Cl. 514/227.2; 514/652; 514/620; 514/415; 514/629; 514/236.2; 514/312; 514/411; 514/653; 514/538; 514/605; 514/456; 514/362; 514/634; 514/567; 514/401

(57) ABSTRACT

The present disclosure provides methods and compositions for treating Impulse Control disorders including, for example, pathological gambling using α2-adrenergic agonists, β-adrenergic receptor antagonists, or both.

### STATEMENT REGARDING FEDERALLY SPONSORED RESEARCH

The invention was made with U.S. government support under grant number DA 17959 awarded by the National Institute of Drug Abuse (NIDA).

### FIELD OF THE INVENTION

This invention generally relates to the treatment of mental disorders. Specifically, the invention provides treatments for psychiatric diseases including impulse control disorders.

### BACKGROUND OF THE INVENTION

Impulse Control Disorders (ICDs) are a sub-group of mental/psychiatric disorders which are characterized by harmful behaviors acted out in response to seemingly irresistible impulses. In the Diagnostic and Statistical Manual of Mental Disorders (DSM-IV), the essential feature characterizing ICDs is the failure to resist an impulse, drive, or temptation to perform an act that is known to be harmful to the actor or others. The impulsive phase (pre-action phase) of an ICD is generally associated with feelings of arousal and/or tension. The impulsive action typically causes these feeling to abate and be replaced with feelings of pleasure and/or gratification.

It is hypothesized that ICDs may be related to or a subset of the obsessive compulsive disorders. Alternatively, or in addition to these psychopathologies, ICDs also frequently have an affective component. Specifically, ICDs often show at least one psychological abnormality common to major depression. ICDs include, for example, binge eating disorders, intermittent explosive disorder (IED), kleptomania, pathological gambling, pyromania, tricholtillomania, compulsive shopping/buying/spending, repetitive self-mutilation, nonparaphilic sexual addictions, severe nail biting, compulsive skin picking, personality disorders with impulsive features, attention deficit hyperactivity disorder, and substance use/abuse disorders.

Gambling is defined in lay terms as placing something of value at risk in the hopes of gaining something of greater value. In the form of sporting events, state-run lotteries and stock market (to name a few) gambling has long permeated modern life, becoming both integral and ubiquitous element of entertainment, business and social activities in our society. While for most persons various forms of gambling remain exciting and enjoyable experience without or with minimal adverse effects, for a substantial minority gambling is acutely reinforcing and profoundly addicting (APA, 2000), leading to seriously maladaptive behaviors culminating in financial collapses, ruined relationships, divorces, increased rates of crime, violence and attempted suicide in 17-24%. These anti-social behaviors and self-destructive tendencies are hallmarks of pathological gambling.

Pathological gambling (PG) is an ICD that also has characteristics of a non-pharmacological addiction; sharing key characteristics with abuse and dependence on pharmacological substances including tolerance, withdrawal, loss of control, unsuccessful attempts to quit, preoccupation, illegal activities, and forfeiting of (social/occupational) responsibilities. The estimated cost of PG to society is about $54 billion; roughly half the cost of substance use disorders or obesity-related problems. Lifetime prevalence of PG in adults is about 5%, but is higher in males and adolescents.

Currently, there is a dearth of safe and effective treatments for PG and other ICDs. Many of the available treatments are ineffective, expensive, experimental and/or have serious deleterious effects that limit the dose or duration of therapy. Furthermore, many available pharmacological treatments are, themselves, prone to addiction and abuse. It is an object of the invention to provide a useful treatment for PG and other impulse control disorders.

### SUMMARY OF THE INVENTION

This invention provides methods and compositions for treating an Impulse Control Disorder (ICD) (e.g., pathological gambling). The invention is based on the discovery that behaviors associated with ICDs are centrally-regulated by nor-adrenergic-dependent pathways and that ICD symptoms may be alleviated by inhibiting nor-adrenergic neurotransmission. Specifically, nor-adrenergic neurotransmission may be inhibited by blocking post-synaptic signal transduction (e.g., through β-adrenergic receptor-dependent mechanism) or by pre-synaptic inhibition of neurotransmitter release (e.g., through α₂-receptor stimulation).

In one aspect, the invention provides a method for treating an ICD in a subject, by administering a therapeutically effective amount of a β-adrenergic antagonist. The β-adrenergic antagonist may be administered alone or in combination with other neuroactive or non-neuroactive agents. Other suitable neuroactive agents include, for example, agents useful for treating an ICD (e.g., an α₂ agonist). Specifically excluded from this combination is the combinations of a β-adrenergic antagonist with a nor-adrenaline (nor-epinephrine) reuptake inhibitor. Non-neuroactive agents may be co-administered with the β-adrenergic antagonist in order to treat a medical condition that is not an ICD.

In another aspect, the invention provides a method for treating an ICD in a subject, by administering a therapeutically effective amount of an α₂ agonist.

In another aspect, the invention provides a method for treating an ICD in a subject, by administering a therapeutically effective amount of an α₂ agonist and a β-adrenergic antagonist. The α₂ agonist and the β-adrenergic antagonist may be administered in the same or different pharmaceutical formulations. When administered in separate formulations, the α₂ agonist and a β-adrenergic antagonist may be administered simultaneously, at different times, with different frequencies, and/or in different dosages.

In preferred embodiments of any of the foregoing aspects of the invention, the ICD being treated is selected from any of the binge eating disorders, intermittent explosive disorder (IED), kleptomania, pathological gambling, pyromania, tricholtillomania, compulsive shopping/buying/spending, repetitive self-mutilation, nonparaphilic sexual addictions, severe nail biting, compulsive skin picking, personality disorders with impulsive features, attention deficit hyperactivity disorder, or substance use/abuse disorders.

In another aspect, the invention provides a pharmaceutical composition containing (i) a β-adrenergic antagonist and (ii) an α₂ agonist. Preferably, the composition is formulated for injection (e.g., intravenous, intramuscular, or subcutaneous) or oral administration. The amount of the β-adrenergic antagonist and/or an α₂ agonist are in an amount sufficient for treating an ICD in a subject.

In other preferred embodiments of the invention, the β-adrenergic antagonist inhibits the biological activity of the β₁- or the β₂-adrenergic receptor. Suitable β-adrenergic antagonists include, for example, propranolol, metoprolol, atenolol, nadolol, pindolol, labetalol, acebutolol, timolol, betaxolol, carteolol, carvediol, oxprenolol, nebivolol, sotalol, pronethalol, alprenolol, esmolol, butoxaminer, and ritodrine.

Suitable α₂ agonists include, for example, clonidine, guanfacine, lofexidine, methyldopa, guanabenz, tizanidine, and xylazine.

Any of the foregoing methods of treatment may be used alone or in combination with non-pharmacological therapies including, for example, psychiatric or other counseling.

By “Impulse Control Disorder (ICD)” is meant any neuropsychiatric disorder characterized by a failure to resist urges to engage in self-destructive behavior. ICDs include, for example, binge eating disorders, intermittent explosive disorder (IED), kleptomania, pathological gambling, pyromania, tricholtillomania, compulsive shopping/buying/spending, repetitive self-mutilation, nonparaphilic sexual addictions, severe nail biting, compulsive skin picking, personality disorders with impulsive features, attention deficit hyperactivity disorder, and substance use/abuse disorders.

By “β-adrenergic antagonist” is meant any compound that has affinity for, and inhibits the biological activity of any variant of a post-synaptic β-adrenergic receptor (e.g., the β₁, β₂, and β₃ adrenergic receptors (ADRB1, ADRB2, and ADRB3, respectively). β-adrenergic antagonists inhibit biological activity through direct binding interactions (e.g., competitive or non-competitive) with the β-adrenergic receptor. Preferably, a β-adrenergic antagonist inhibits a β-adrenergic receptor with an IC₅₀ of less than about 1 μM, less than about 100 nM, less than about 10 nM, or less than about 1 nM. Also preferably, the β-adrenergic antagonist inhibits a β₁ or β₂ adrenergic receptor.

By “α₂ agonist” is meant any compound that has affinity for, and activates the biological activity of a presynaptic α₂-adrenergic receptor. Preferably, an α₂ agonist activates presynaptic α₂-adrenergic receptor with an EC₅₀ of less than about 1 μM, less than about 100 nM, less than about 10 nM, or less than about 1 nM. Preferably, an α₂ agonist activates at least one of the α_{2A}, α_{2B}, or α_{2C} subtypes of the α₂-adrenergic receptor (also known as the ADRA2A, ADRA2B, and ADRA2C subtypes, respectively).

By a “therapeutically effective amount” is meant a quantity of compound (e.g., an α₂ agonist, β-adrenergic antagonist, or a combination thereof) that when delivered with sufficient frequency provides a medical benefit to the patient. Thus, a therapeutically effective amount of a compound in a dosage form sufficient to treat or ameliorate one or more symptoms of an ICD.

By “pharmacological treatment” is meant administering a pharmaceutical composition for the purpose of improving the condition of a patient by reducing, alleviating, or reversing at least one adverse effect or symptom. It is recognized that ICDs may be treated according to the principles of this invention using pharmacological treatment alone or in combination with other non-pharmacological treatment modalities including, for example, psychiatric counseling, participation in support groups or other forms of group therapy, and hypnosis.

By “neuroactive agent” is meant any compound that is administered to an individual for the purpose of therapy whose primary mechanism of action is mediated within the central nervous system, or is administered for the purpose of alleviating symptoms of a brain disorder (e.g., a psychiatric disease).

### BRIEF DESCRIPTION OF THE DRAWINGS

FIG. 1 is a schematic diagram illustrating a proposed mechanism for cross-sensitization between drug reward stimuli and stress.

FIG. 2 are a pair of images showing equivalent brain slices from (A) the MP-RAGE3D and (B) T1-weighted match warped image series which reflects the actual geometry of the functional scans.

FIG. 3 is a series of images showing the brain activity of a patient during the expectancy phase for cocaine infusion (top row) and during the actual cocaine infusion (bottom row).

FIG. 4 is a schematic diagram of three different “spinners” used in the monetary reward studies. Depicted are the “bad” spinner (highest probability of losing money), the “intermediate” spinner (moderate likelihood of winning money), and the “good” spinner (highest likelihood of winning money).

FIG. 5 is a bar graph showing the subjective expectancy and satisfaction ratings by PTSD and non-PTSD patients during a gambling task using the spinners depicted in FIG. 4.

FIG. 6 is a line graph showing the subjective expectancy ratings of healthy and cocaine-addicted subjects during a gambling task using the spinners depicted in FIG. 4.

FIG. 7 is a series of line graphs quantifying the fMRI signal from various brain regions from a cocaine-addicted subject during the expectancy and outcome phases of a gambling task.

FIG. 8 is a series of group activation fMRI maps demonstrating differences in brain activity between PTSD and non-PTSD subjects during a gambling task.

FIG. 9 is a series of coronal fMRI slices from a healthy subject showing positive activation in the NAc and amygdala in response to rewarding stimuli and a reduced signal in the NAc in response to stressful picture stimuli.

FIG. 10 is a series of coronal fMRI slices from a PTSD patient showing that activation of the lateral prefrontal cortex (LPC), amygdala, hippocampus, and periaqueductal gray/ventral tegmental regions (PAG/VT) are sensitized (more activated) in PTSD patients in response to aversive stimuli.

FIG. 11 is a schematic diagram of the two monetary reward spinners and ratings slider used in Example 6.

FIG. 12 is a series of bar graphs showing (A) the spinner choices, (B) the regret level, and (C) the expectancy and satisfaction ratings of alcohol-dependent, heroin-dependent, occasional alcohol/heroin use, and healthy subjects as described in Example 6.

FIG. 13 is a series of fMRI slices from a patient administered saline or cocaine. The slices show the subcortical brain regions and demonstrate significant fMRI signal changes after cocaine, but not saline, infusions.

FIG. 14 (left panel) is a series of KS maps showing activation in the right and left NAc following morphine administration compared to saline controls. The data is averaged for 5 subjects. FIG. 14 (right panel) is a series of line graphs showing the time-course of activation of the left NAc by morphine and saline. Data is the mean of 5 subjects and percent signal change is normalized relative to each subject's pre-infusion baseline, but not detrended.

### DETAILED DESCRIPTION OF THE INVENTION

The present disclosure provides methods and compositions useful for treating Impulse Control Disorders (ICDs) and, particularly, pathological gambling. ICDs are treated using therapeutically effective amounts of β-adrenergic antagonists, α₂ agonists, or both. Optionally, one or more neuroactive therapeutic compounds is included.

Impulse Control Disorders (ICDs) are a diverse group of neurological/psychiatric disorders that are linked by a failure to resist urges to engage in self-destructive behavior. This behavior may be destructive to the financial, social, or medical well-being of the patient. ICDs that are socially or financially destructive are often the most difficult to diagnose at the earliest stages.

Pathological gambling (PG) is one particularly destructive type of ICD. A consistent clinical finding in PG is an exaggerated sympathoadrenal tone suggestive of heightened levels of stress and arousal as evidenced by increased heart rate, increased plasma and CSF nor-adrenaline concentrations, and increased skin conductance levels at baseline and during gambling activities. These physiological alterations, together with sensitized brain metabolic reactions to gambling, create a cross-sensitization phenomenon similar to that observed by others in substance use disorders and stress. Indeed, the sensitized stress responses in PG are mostly conspicuous in the context of gambling and gambling-related cues; whereas, stress is a key factor responsible for the chronically relapsing nature of PG.

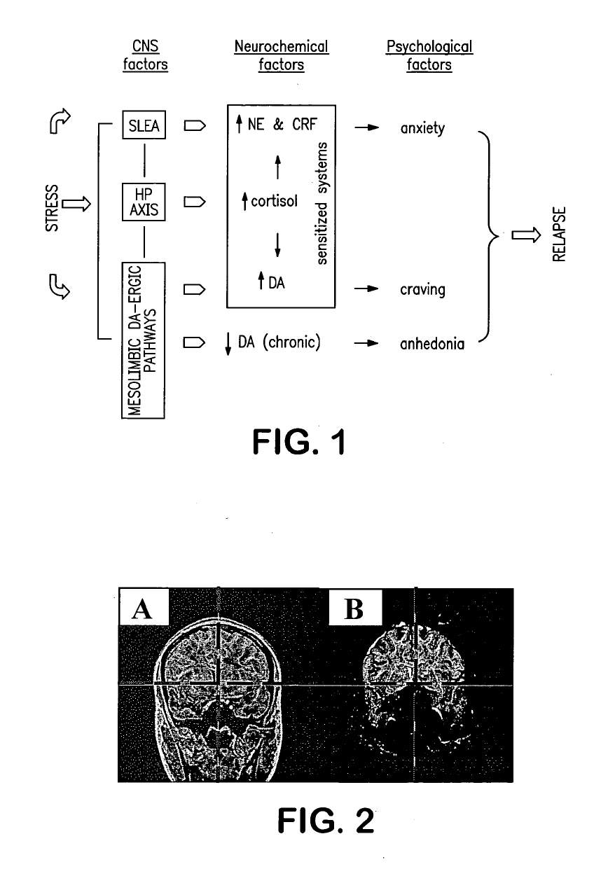

Cross-sensitization is a multifactorial process that encompasses several neurochemical, neuroanatomical, and functional systems including the mesolimbic dopaminergic pathways, noradrenergic and corticotrophin releasing factor (CRF) neurotransmission within the sublenticular extended amygdala (SLEA) structures (esp. the central nucleus of the amygdala and the bed nucleus of the stria terminalis (BNST)), and the hypothalamic-pituitary-adrenal axis. These three systems are infused with a variety of inter-related glutamatergic, GABAergic, opioidergic, and serotoninergic neurons and pathways. For example, FIG. 1 illustrates one possible mechanism for drug/stress cross-sensitization that is responsible for relapse. Thus, in order to treat PG and other ICDs, the pathways at the interface of reward, reinforcement, and stress must be identified and targeted for pharmacologic intervention.

Dopaminergic Reward Pathways: Mesolimbic dopaminergic pathways projecting from the ventral tegmentum (VT) to the nucleus accumbens (NAc), amygdala and medial prefrontal cortex (mPFC) are responsible for the incentive motivational aspects of reward function. These are collectively termed “wanting processes” and include conditioned learning of stimulus-reward association, reward prediction, and attribution of incentive salience to rewarding stimuli.

Sensitization and Tolerance in Substance Use Disorders (SUDs): Drug-induced changes in the mesolimbic dopaminergic circuitry underlie the wanting, but not liking purposes are responsible for transforming regular wanting responses into heightened incentive salience assigned to drugs or drug-related cues. This incentive sensitization process in construed to be an animal homolog of human craving. A closely related concept, derived from primate work, is the aberrant learning theory suggesting that learning of new rewards is encoded via interactions between tonic (baseline) and phasic spikes in dopaminergic neurons, in which phasic firing predicts new rewards. Therefore, neural adaptions to excessive dopaminergic trafficking in response to drugs leads to an “over-learning” of the motivational significance of cues that predict delivery of drugs.

Stress is Involved in Both Sensitization and Tolerance: Acute stress activates dopaminergic neurotransmission in the same dopaminergic reward pathways. Chronic stress exerts an opposite action by decreasing dopaminergic neurotransmission and is accompanied by decreased motivation towards normally pleasurable stimuli. At the same time stress causes sensitization in the form of stress-induced cravings. Cortisol, a stress hormone, appears to enhance the salience of drugs and drug-related stimuli along with dopaminergic neurotransmission within the reward circuitry. Furthermore, chronic stress-related cortisol elevations contribute to the sensitization of the extrahypothalamic CRF system. Specifically, recurrent stress exposure causes noradrenaline and consequent CRF hypersecretion within the SLEA structures which underlies the feelings of anxiety and fear.

Sensitization and Tolerance in PG Symptomatology: Poor impulse control is considered a key component in PG. However, recent work suggests that PG may also be classified as a “reward-deficiency” syndrome. In addition to a high comorbidity with substance use disorders, there are clinical and diagnostic indicia that suggest a reward system dysfunction. Most notable are tolerance, withdrawal, and sensitization. In the context of PG, tolerance is characterized by the urge to gamble with increasing amounts of money in order to achieve the desired effect. Withdrawal typically is characterized by restlessness and irritability during periods of no gambling and sensitization is an increased preoccupation with gambling. The latter is evident in neuroimaging studies that reveal increased activity in the frontal and striatal regions accompanied by increased gambling urges in response to gambling cues or during actual gambling.

Table 1 summarizes the results of several recent neuroimaging studies from patients diagnosed as being pathological gamblers. Patenza et al. reported decreased blood flow in the similar areas during gambling cues accompanied by increased gambling urges. The comparison between this (Potenza et al.) and the two other studies (Crockford et al, 2005; Hollander et al, 2005) may be somewhat complicated by combining of emotional and gambling cues in a block-like design in the Potenza al (2003a) study. Notably, hypofrontality or decrease in cerebral blood flow (CBF) when challenged by a cognitive task along with limbic hyperactivity during gambling stimuli closely resemble the neuroimaging observations recorded in the SUDs patients at baseline/throughout cognitive challenges, or when exposed to drugs or to drug-related cues. Also, similar to diminished drug-induced activations in SUDs (i.e., tolerance), pathological gamblers displayed decreased NAc responses to monetary gains.

| Authors | Subjects | Stimulus | Technique | Findings |
|---|---|---|---|---|
| Crockford | patients | visual | fMRI | PG patients displayed increased activity |
| et al, | with PG | gambling cues | | in the rt. inferior frontal gyrus & rt. right |
| 2005 | (N = 13) | | | medial frontal gyrus along with cues- |
| | healthy | | | induced craving for gambling |
| | controls | | | |
| | (N = 10) | | | |
| Hollander | patients | blackjack: | ¹⁸F-DG | monetary reward > computer points in |
| et al, | with PG | monetary | PET | mPFC, striatum and visual cortex |
| 2005 | (N = 7) | reward vs. | | |
| | | computer | | |
| | | points | | |
| Reuter et | patients | choosing a | fMRI | PG patients displayed decreased activity |
| al, 2005 | with PG | winning vs. | | in the rt. NAC and mPFC during the |
| | (N = 12) | losing card, | | gambling outcome phase, negatively |
| | healthy | accompanied | | correlated with severity of gambling. |
| | controls | by monetary | | |
| | (N = 12) | gains and | | |
| | | losses, | | |
| | | respectively | | |
| Potenza | patients | videotapes | fMRI | decreased activity in frontal, striatal & |
| et al, | with PG | with happy & | | thalamic regions in PG prior to subjective |
| 2003a | (N = 10) | sad content & | | gambling urges |
| | healthy | with | | increased occipital activation in PG with |
| | controls | gambling- | | the onset of gambling urges |
| | (N = 11) | related content | | increased frontal & ventral striatal |
| | | interspersed | | activations in PG in response to happy |
| | | with emotional | | stimuli |
| | | stimuli | | decreased rt. inferior frontal gyrus |
| | | | | activity in PG during sad stimuli |
| Potenza | patients | Stroop task | fMRI | decreased PFC activity in PG |
| et al, | with PG | | | |
| 2003b | (N = 13) | | | |
| | healthy | | | |
| | controls | | | |
| | (N = 11) | | | |

### Treatment of PG and Other ICDs

There is a cross-sensitization between the consumption of addictive drugs and stress. Further, there are similar neurobiological mechanisms and pathways underlying SUDs and PG. There is also a similar cross-sensitization between stress and PG.

Thus, PG and other ICDs may be treated by inhibiting the cross-sensitization that occurs among the dopaminergic, noradrenergic, and CRF pathways in the brain. Treatment involves administering a pharmacologically effective amount of a compound that is centrally active (i.e., able to cross the blood-brain-barrier) and inhibit noradrenergic neurotransmission. Specifically, ICD treatment is effected by administering either or both of an α₂ agonist and a β-adrenergic antagonist in an amount and manner sufficient to alter central nor-adrenergic neurotransmission.

The α₂-adrenergic receptors, including the α_{2A}, α_{2B}, α_{2C} subtypes, are well-known G-protein coupled receptors. Centrally, the α₂ receptors are pre-synaptic and activation results in reduced cell firing and concomitant release of noradrenaline from the presynaptic terminals, which is mediated by the hyperpolarizing effect of an inwardly rectifying K⁺ conductance. Suitable α₂ agonists include, for example, clonidine (0.05-5.0 mg/day), guanfacine (1-10 mg/day), lofexidine (0.2-10 mg/day), methyldopa (250-5000 mg/day), and guanabenz (4-150 mg/day).

The β-adrenergic receptors, including the β₁, β₂, and β₃ subtypes, are also G-protein coupled receptors. These receptors are predominantly post-synaptic so β-adrenergic antagonists are required to disrupt noradrenergic neurotransmission. Suitable β-adrenergic antagonists include, for example, propranolol (10-1000 mg/day), metoprolol (25-1000 mg/day), atenolol (25-1000 mg/day), nadolol (20-1000 mg/day), pindolol (5-300 mg/day), labetalol (100-3000 mg/day), acebutolol (200-3000 mg/day), timolol (5-100 mg/day), betaxolol (10-200 mg/day), carteolol (2.5-100 mg/day), and carvediol (12.5-500 mg/day).

It is recognized that co-administration of any therapeutics described herein may be administered individually (i.e., at different dosages with different frequencies, durations, and/or routes of administration). The doses provided above are merely guidelines for administration and treatment of an ICD and should not be construed to be limiting. The attending physician will select the appropriate drug, frequency, dosage, route of administration, and duration of therapy. It is contemplated that any therapy administered to treat an ICD according to the principles of this disclosure will be titrated to achieve the maximal effect with minimal/acceptable side-effects. The therapy is likely to vary on an individual-by-individual basis. Further contemplated within the scope of this invention is the co-administration of any other neuroactive compound for the treatment of the same ICD, another ICD, or any other co-morbid condition.

### Example 1

### Improved Localization of fMRI Activation in the Basal Forebrain

Because NAc is a primary ROI, we sought to refine its (and other regions) visualization on the functional scans. Other key components of reward circuitry, including VT, NAc, amygdala and mPFC, are located in the regions of significant magnetic field inhomogeneity caused by nearby air-tissue interfaces in the sinuses and mouth. In echoplanar images (EPI; as used in fMRI) this results in signal reduction and severe non-rigid body deformation of images, a problem that is more pronounced in high field (≧3T) scanners. fMRI images are of low resolution and contrast, so activations are mapped onto to high resolution anatomic scans. However, the mapping between standard anatomic and fMRI images is spatially variable in the presence of inhomogeneities, and activations in the basal forebrain are quite displaced relative to the high resolution images. Spatial transformations, such as Talairach warping, could magnify this problem. Match warped images are high resolution images acquired using the same parameters that cause exactly the same distortion as echoplanar imaging. Although distorted, the high resolution and signal to noise ratio of these images permit easy identification of neuroanatomical structures. Activations and identifiable anatomic structures may be exactly overlaid, even in regions of severe spatial distortion. To address this issue two sets of matching images were collected (N=4): the first is a standard T1 weighted high resolution MP-RAGE3D acquisition. In addition, “match warped” images (68 T1 weighted coronal slices, FOV=220 mm, 256×256, 3 mm thick) were collected using the technique developed by Dr. Frederick (Frederick et al, 2004). The NAc's positioning error was 11.2±6.2 mm vs. 4.7±2.9 mm for anatomical landmarks in more homogeneous field regions. The images shown in FIG. 2 demonstrate this phenomenon in equivalent slices from an MP-RAGE3D and TI-weighted match warped image series. In FIG. 2, note that the area around the ventricles and the NAc is displayed upwards in the warped image by about 1 cm, while the sulci in the lateral brain are aligned with the MP-RAGE3D image.

### Example 2

### Multimodal Assessment of Reward Function in SUDs

This study used behavioral probes to assess: a) whether incentive sensitization for drugs spills over to non-drug rewards, and b) whether patients with SUDs are more sensitive to stress in comparison to healthy subjects.

To address these questions, non-drug psychosocial and biochemical probes of reward function were administered to the same four groups of male participants with alcohol (N=19; age=34.5±1.16) and heroin (N=20; age=28.1±1.11) dependence along with occasional alcohol/heroin users (N=20; age=27.6±0.7) and healthy controls (N=24; age=27.1±1.07). Four distinct experimental paradigms employed in this project included: a) sucrose solutions administered in the context of the sweet preference test, social reward tasks in the form of visual processing of b) attractive vs. average faces and c) positive vs. aversive images (IAPS), and d) monetary incentive stimuli incorporated into a gambling task.

For the key-press task, it was explained to the subjects that they could keep the viewing time at 8 s for a facial or positive/aversive image by not pressing any computer key, or either increase or decrease this time by up to 4 s (depending upon the frequency of the key presses) by alternately pressing a keyboard's “n” and “m,” or “z” and “x” keys, respectively. The former key presses were scored as positive and the latter as negative. The average of these values for the 20 pictures in each of the four facial or the images categories yielded a subject's “net” key presses for each category. In addition, each subject's total key presses, i.e., absolute number of key presses, regardless of whether scored positive or negative, during the entire experiment was calculated for use as a covariate. During the subsequent rating task, the subject rated the attractiveness of the same images on Likert-type scales ranging from 1 (“very unattractive”) to 12 “very attractive.” The averages for the 20 pictures in each of the four facial and three images categories yielded a subject's attractiveness rating for each images' category.

We found that: 1) SUDs subjects had higher preference for sweet solutions than healthy controls i.e, reward sensitivity. The minimal concentration of sucrose (0.05 M) was preferred by 14.3% of subjects with alcohol dependence, 16.7% of subjects with heroin dependence, 36.4% of occasional alcohol/heroin users and 54.2% of healthy controls (p=0.015, Fisher's Exact Test). Conversely 19.0% of subjects with alcohol dependence, 50.0% of subjects with heroin dependence, 4.5% of occasional alcohol/heroin users and 8.3% of healthy controls preferred the highest sucrose concentration (0.83M; p=0.002, Fisher's Exact Test); 2) SUDs subjects displayed higher motivation (in the units of key presses) for beautiful faces and positive images. The average number of key presses for the attractive female images and for positive images, respectively were 25.4±0.5 and 6.4±0.25 for subjects with alcohol dependence, 29.6±0.4 and 9.5±0.27 for subjects with heroin dependence, 30.0±0.4 and 6.4±0.25 for occasional alcohol/heroin users and 25.9±0.35 and 5.4±0.23 for healthy controls (p<0.001 for type of images by group interaction by ANCOVA, with total key presses as the covariate); 4) The attractiveness ratings generally paralleled the keypress data and 5) Increased stress sensitivity in the SUDs group was evidenced by greater effort (in the units of computer key presses) exerted by these subjects to get rid of the negative images i.e., −5.7±0.3 for subjects with heroin dependence, −5.9±0.33 for occasional alcohol/heroin users and −4.0±0.43 for healthy controls (p<0.01). Additionally, SUDs subjects made fewer gambling risky stakes: 21.8±0.69 for subjects with alcohol dependence, 26.6±0.69 for subjects with heroin dependence, 23.08±0.69 for occasional alcohol/heroin users and 31.3±0.63 for healthy controls.

Taken together these findings supports the concepts of spillover of incentive sensitization to non-drug rewards (e.g., sweets, beauty and positive images), generalizability of this phenomenon to dependence on various classes of addictive substances such as alcohol and heroin and drugs-stress cross-sensitization. Given that PG may be “addiction without exogenous substance use,” these data suggest that similar spillover sensitization occurs in PG patients.

### Example 3

### Expectancy Effects Versus Pharmacological Effects

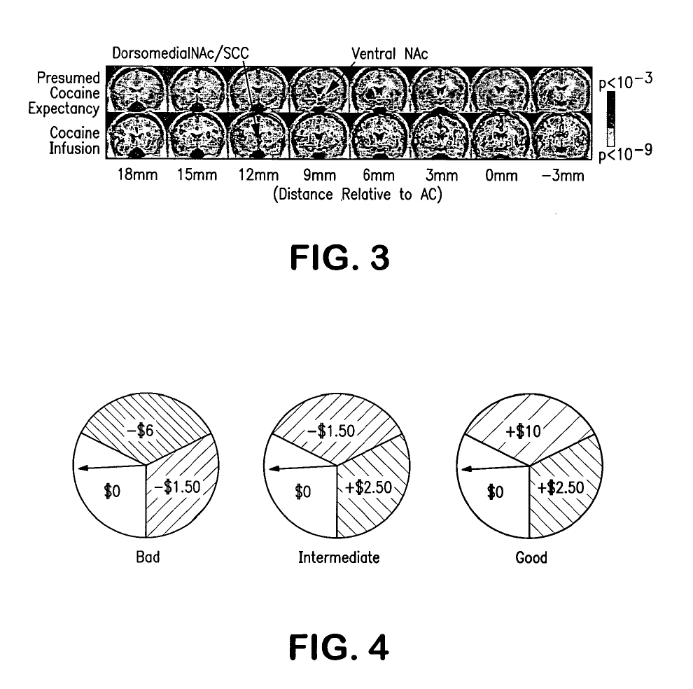

It is believed that the VT to NAc circuitry is involved with the prediction of rewarding events. To evaluate this possibility, we analyzed the pre-infusion baseline before the anticipated cocaine infusion. Both individual analysis, and analysis of averaged data, revealed pre-infusion activation of the ventral region of the NAc (FIG. 3), approximating the shell region of the NAc in primates and humans to which project medial VT neurons involved with reward prediction. This activation occurred prior to both the cocaine and saline infusions, i.e., a 50% expectancy condition. Notably, this ventral region of the NAc did not activate in response to cocaine infusion, and subjects reported no consistent concurrent subjective effects of rush, high, low, or craving. These data demonstrate the dissectability of reward function into expectancy and outcome domains and underscores the heuristic value of the type of the monetary stimulus paradigms proposed in this application.

### Example 4

### Monetary Stimulus

In the post hoc analyses of cocaine infusion data in cocaine dependent subjects (Example 3), activation of the NAc had a temporal component, in its ventral extent, that showed signal changes preceding any infusion, and then continuing briefly after both cocaine and saline infusions for 1-3 minutes before returning to baseline. This ‘expectancy’-like activation in the NAc led us to consider a nonpharmacological reward stimulus whereby expectancy effects could be dissociated from the outcome (gains/losses) effects.

Money, an easily quantifiable reward, valued by most people, provides a rich framework for cognitive neuroscience research. It is also an ecologically valid stimulus to be used in patients with gambling problems. Functional neuroimaging studies using financial incentives have associated monetary reward with neural response in the NAc/ventral striatum (including dopamine release measured by ¹¹C raclopride), SLEA, amygdala and mPFC. Notably, it has been shown in subjects dependent on opioids or tobacco, that the receipt of monetary reward evoked reduced ventral striatum activation in comparison to healthy controls.

During the gambling paradigm, subjects participated in a game of chance where they actually won or lost money while in the fMRI scanner. Signal changes that anticipate or accompany monetary gains and losses under varying conditions of controlled expectation were evaluated. Specifically, evaluated were: a) an “expectancy phase”, when a promising or unpromising roulette-like spinner (FIG. 4) is presented and b) an “outcome phase” when the arrow lands on one sector of that spinner, and thereby alters the subjects' winnings. It is known that, when applied to healthy controls, this task differentiates the fMRI time courses in the NAc, mPFC and SLEA and in related regions during expectancy vs. outcome phases.

At the start of the game, each subject was given an endowment of $50 and was told that (s)he might lose some or all of this stake, retain it or increase it. Each trial consisted of a “prospect phase”, when a spinner was presented and an “outcome phase” when a sector of the spinner was selected by the arrow and a corresponding amount was added to or subtracted from the subjects' winnings. During the prospect phase the image of one of the spinners was projected for 6 seconds and the subject pressed one of four buttons to identify the displayed spinner, thus providing a measure of vigilance. The display was static for the first 0.5 second and then a superimposed arrow began to rotate. The arrow came to a halt at 6 seconds marking the end of the prospect phase. During the first 5.5 seconds of the ensuing outcome phase, the sector where the arrow had come to rest flashed, indicating the outcome. A blank screen was then projected during the last 0.5 seconds of the 12-second trial. On fixation point trials an asterisk appeared in the center of the display for 15.5 seconds followed by 0.5-second blank screen. The trial sequence was not truly random but rather was fully counterbalanced so that trials of a given type (spinner+outcome) were both preceded and followed once by all nine spinner/outcome combinations and three times by fixation point trials. Sixteen sets of runs with 19 trials each were presented to subjects. Only results of the last 18 trials were scored for each run since the initial trial was inserted into the run sequence purely to maintain counterbalancing. Runs were separated by 2-4 minute rest periods. The display presented to the subject consisted of either fixation point (an asterisk) or 1 of 3 spinners. Each spinner (FIG. 4) was divided into three equal sectors. The “good” spinner yielded a large gain (deep green sector labeled +$10), a small gain (light green sector labeled +$2.50) or no gain (white sector labeled $0). The “bad” spinner yielded a large loss (deep red sector: −$6), a smaller loss (light red sector −$1.50) or no loss (white sector $0). The “intermediate” spinner yielded a small gain (light green sector +$2.50), a small loss (light red sector −$1.50), or neither a loss nor a gain (white sector: $0). Gains were greater than losses to adjust for assignment of greater salience to a loss than to a gain of equal amount. The same trial sequence was used for all subjects (unbeknownst to them), generating total winnings of $78.50 to each, added to the $50 endowment.

All images were acquired on a 3 Tesla Siemens Trio MR imaging system; subjects' responses were analyzed with BrainVoyager. An algorithm for acquisition of match warped images developed at McLean Brain Imaging Center was employed to allow exact registration of the functional and anatomic data in the presence of severe magnetic field inhomogeneities.

### 4.1: Gambling Task in PTSD and Non-PTSD Patients

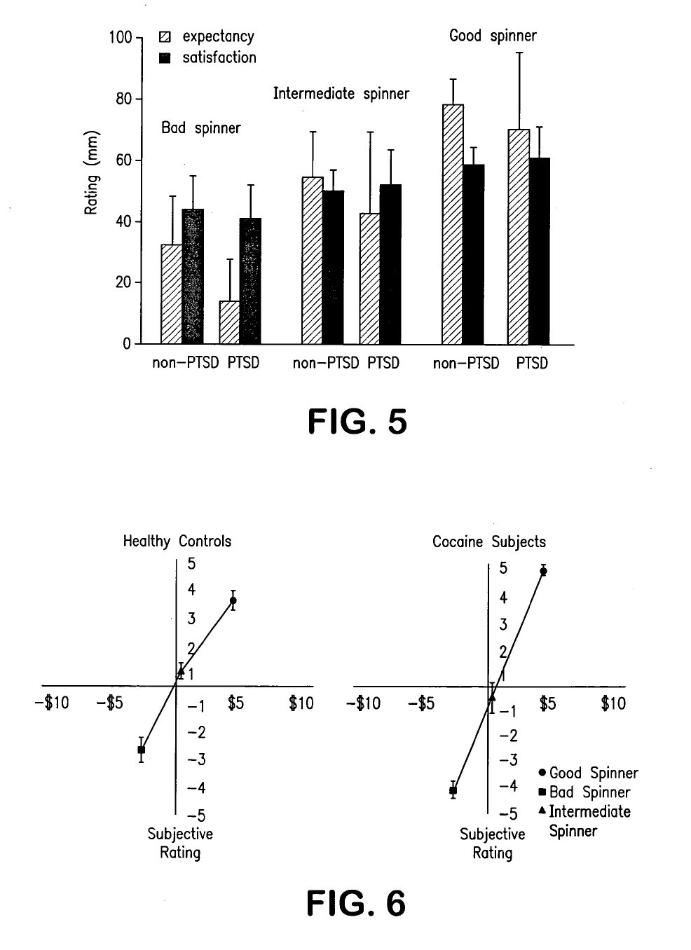

A monetary stimulus task was administered to 13 patients diagnosed as having post-traumatic stress disorder (PTSD; 12 males, 1 female) and 13 trauma-exposed non-PTSD controls (11 males, 2 females). During this test, subjects gave an “expectancy” rating when viewing each spinner before the trial, and then a “satisfaction” rating based on the spinner's outcome. The spinners shown in FIG. 4 were used. One-way ANOVA revealed significantly lower ratings of total expectancy (p<0.05) and expectancy for the bad spinner (p<0.01) in the PTSD vs. non PTSD participants, but no significant group differences in the satisfaction ratings (FIG. 5). The lesser overall expectation in the PTSD group is indicative of their under-responsive reward circuitry.

### 4.2: Gambling Task in Cocaine-Dependent Patients

Self-reported data from cocaine-dependent subjects (n=13) undergoing the same monetary reward task as described above indicate that, relative to healthy controls, addicts have a more extreme range of responses to expectancy information, suggesting they may have overactive expectancy assessment (FIG. 6). Comparison of subjective response to spinners across groups was significant for the good spinner and the bad spinner (p<0.017, unpaired t-test).

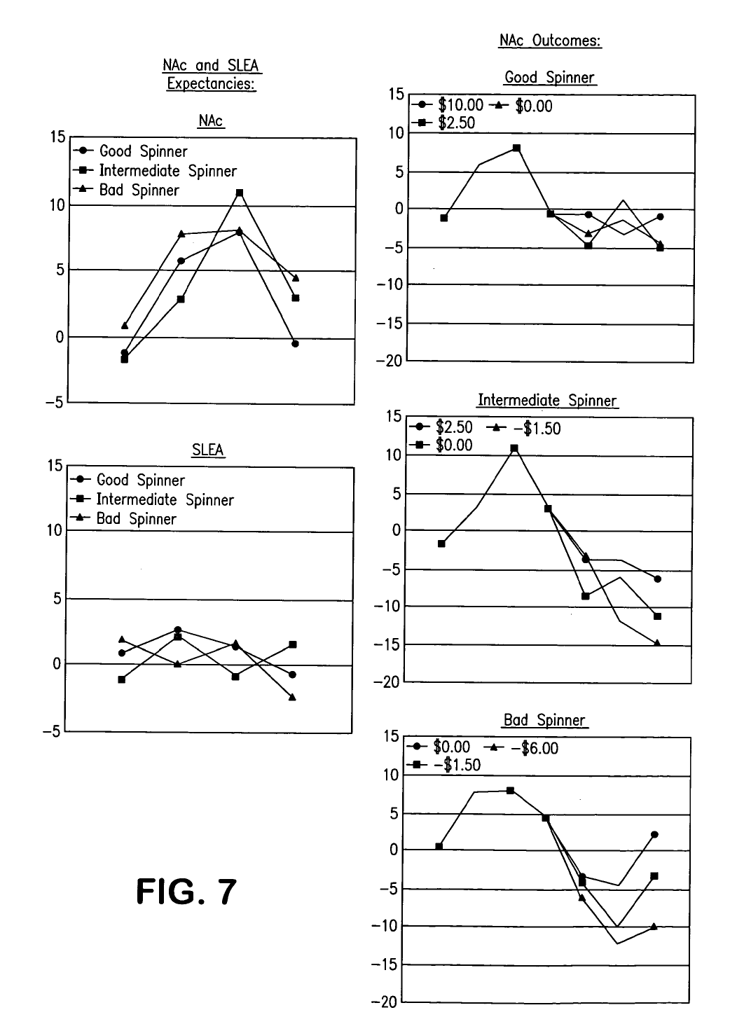

The fMRI data, analyzed to date in one subject, shows stronger activation in regions responding to expectancy information, with subsequent further decrements of fMRI signal during the subsequent outcome phase of the experiment. A 4-5 fold increase in NAc signal change is observed in the cocaine-dependent patient relative to healthy controls (FIG. 7). Also, note the relative absence of a SLEA expectancy effect. The increased NAc signal change during the expectancy phase (timepoints 1-4), and decrement during the outcome phase (timepoints 5-7) suggest an extreme version of expectancy/reward interaction.

### 4.3: Reward Circuitry Changes in PTSD Patients During a Gambling Task

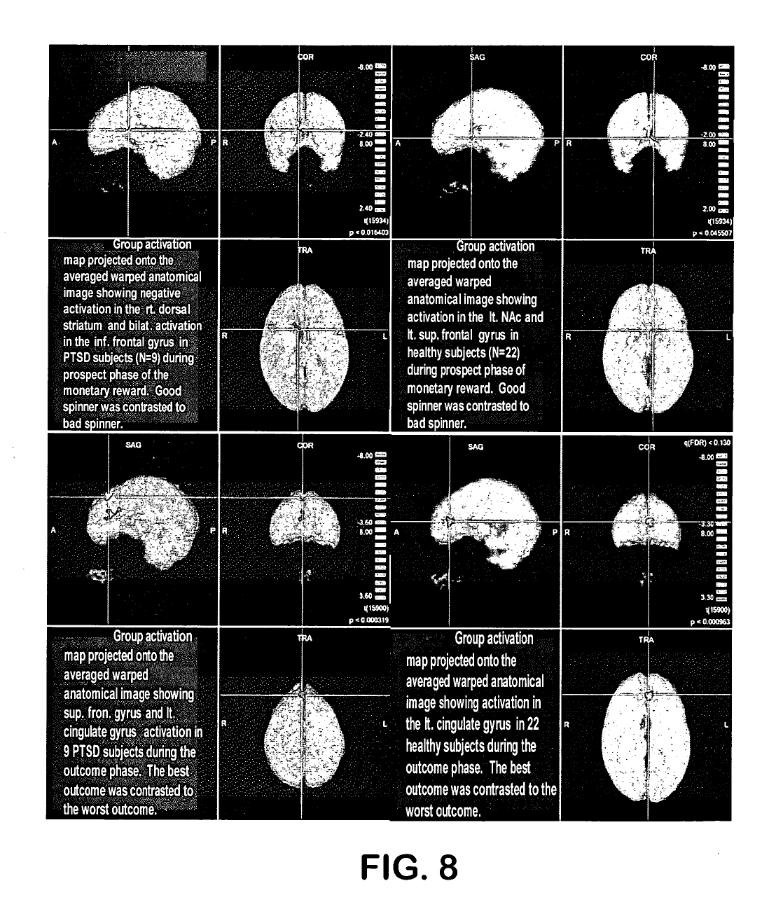

This experiment evaluated local hemodynamic responses using BOLD fMRI that either anticipate or accompany monetary gains and losses under varying conditions of controlled expectation in 9 patients with PTSD and 22 healthy volunteers. As depicted in FIG. 8, patients with PTSD displayed altered activity in the NAc and other reward-related regions (p<0.05, corrected for multiple comparisons). The study demonstrates that expectancy is neurochemically different from actual receipt of reward in a group of psychiatric patients that purportedly have a dysfunctional reward circuitry.

### Example 5

### Social Stimuli (Pleasant and Aversive Images; IAPS)

### 5.1: Healthy Subjects

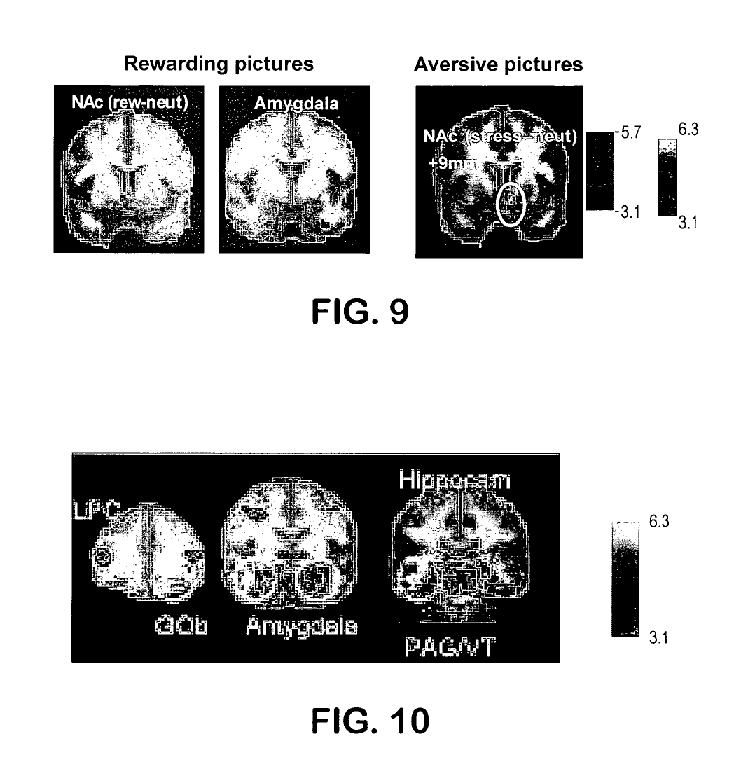

Three healthy male subjects were administered the IAPS protocol. Three sets of 10 aversive/unpleasant, rewarding/pleasant and neutral pictures were shown during acquisition of functional measures in a 3T magnet. Subjects rated (R) their level of distress (1<R<5), reward (5<R<9) and neutral (5) on the IAPS scale of 1-9 at the end of each set. Subjective ratings were consistent with those devised by IAPS in healthy subjects: aversive stimuli=2.1±1; neutral stimuli=5.1±0.2; and rewarding stimuli 7.5±0.4; mean±SD). FIG. 9 shows positive activation in the NAc and amygdala (left panels) to rewarding pictures (Neutral—Rewarding) and decreased activation in the NAc (right panel) to aversive pictures (Neutral—Aversive).

### 5.2: Subjects with PTSD

IAPS was also used to apply rewarding and stressful stimuli to both PTSD patients (N=6) and healthy controls (N=2) while they were imaged in the 3T medical imaging magnet. During each patient's scan session, three functional scans were taken, during which the patient viewed a set of IAPS pictures. For each of the three functional scans a unique set of IAPS images were used. The presentation of images was designed such that three blocks of pleasant images, three blocks of aversive images, and four blocks of neutral images were viewed by the patient, in the same pattern, for each of the 3 functional scans. Importantly, exaggerated (i.e., sensitized) amygdala responsivity to stressful stimuli was observed (FIG. 10).

### Example 6

### Gambling Task in SUDs Patients and Occasional Substance Users

This task utilized heterosexual male participants that were alcohol dependant (n=20; age±SD=33.7±4.64), heroin dependant (n=18; 28.1±4.69), occasional alcohol or heroin users (n=22; 27.6±3.28), or healthy non-users (n=24; 27.1±5.23).

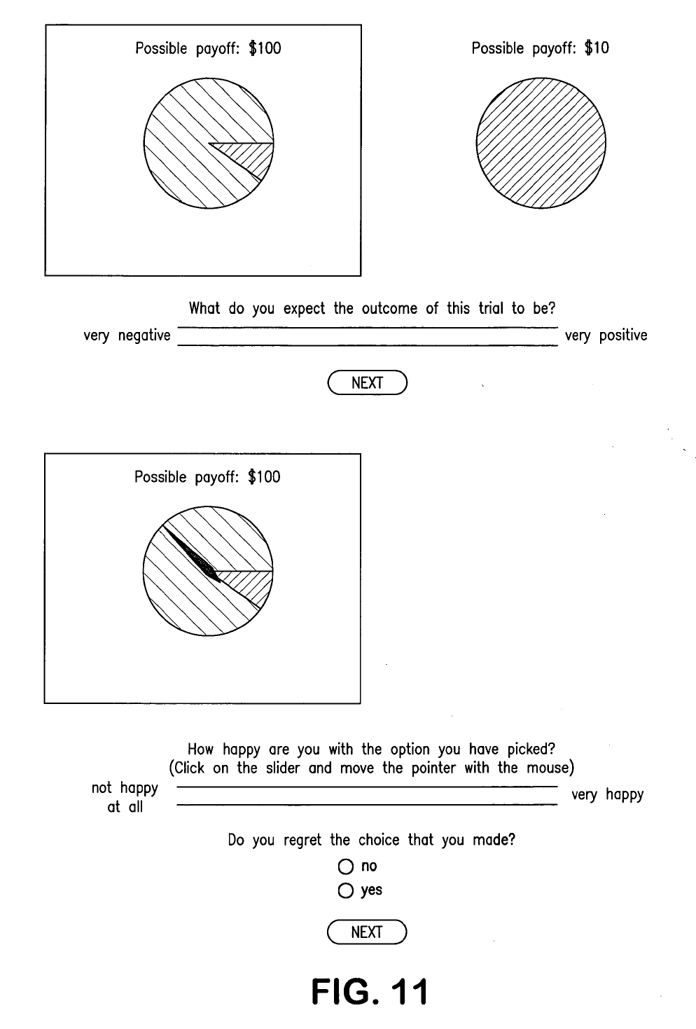

Subjects were probed in a gambling/monetary reward task using the two spinners shown in FIG. 11. Briefly, subjects chose one of the two spinners (high risk/high reward or low risk/low reward) and asked to score there expectancy of a favorable outcome using the slider bar, as shown. This is the “expectancy phase”. Following the trial, subjects were asked to rate their satisfaction with the outcome (“satisfaction phase”). Following the satisfaction scoring, subjects were asked to score their regret for not choosing the other spinner (“regret phase”). In alternate trials, subject were either shown the outcome of the non-chosen spinner (“with counterfactual comparison”) or that outcome was not shown (“without counterfactual comparison).

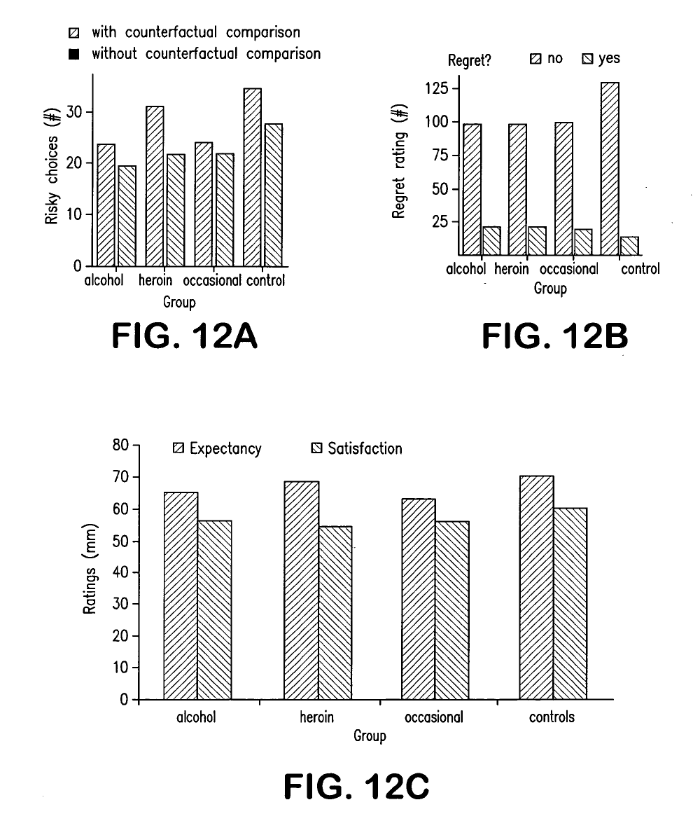

Substance-dependent subjects (alcohol and heroin) and occasional users made significantly less risky choices (FIG. 12A) and expressed significantly more regret about their choices (FIG. 12B). As shown in FIG. 12C, expectancy ratings were dependent upon the group (F=12.8, p<0.001), type of spinner (F=587.6, p<0.001), and interaction between group and type of spinner (F=45.7; p<0.001); covaried for age and adjusted for multiple comparisons. Post-hoc analysis revealed that alcohol dependent subjects and occasional users reported significantly lower expectancy than healthy controls. The satisfaction rating were dependent upon group (F=11.6; p<0.001), type of spinner (F=4.3; p=0.03), and interaction between group and type of spinner (F=6.9, p<0.009); covaried for age and adjusted for multiple comparisons. Post-hoc analysis of the satisfaction data revealed that dependent subjects and occasional users reported significantly lower satisfaction than healthy controls.

Taken together, these data demonstrate that consumption of addictive substances, both dependence and occasional use, is associated with reward tolerance which is reflected in reduced expectancy and satisfaction.

Taken together with the previous examples, individuals exposed to addictive substance are sensitive to the effects of stress reflected in enhanced motivation to get rid of negative images, avoidance of risky gambling choices, and regret about the gambling choices. The data further demonstrates the cross-sensitization between stress mechanisms and the reward pathways of the brain suggesting that pharmacological manipulation of the stress mechanisms can alter reward-seeking behavior. This is particularly important in the treatment of ICDs like PG which have been shown to directly affect the reward centers of the brain.

### Example 7

### Pharmacological Stimuli

### 7.1: Mapping of Subcortical and Brainstem Regions

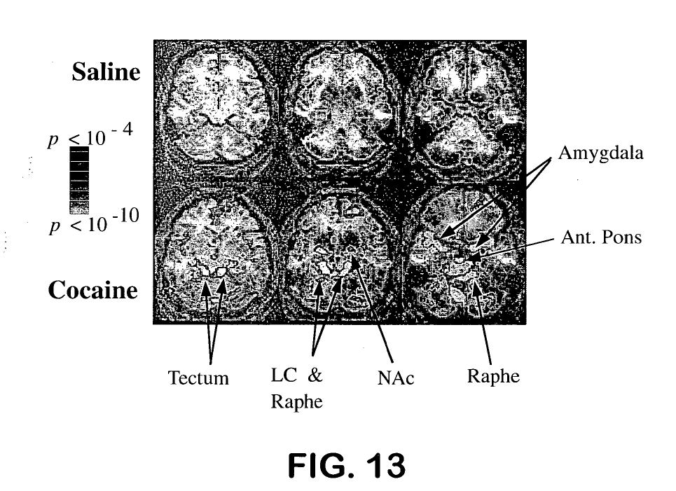

We imaged reward circuitry in humans during cocaine infusion, using cardiac gated fMRI BOLD imaging to compensate for brainstem motion. We further used a clustered volume acquisition with sharpened slice profiles to cluster the acquisition of the brainstem volume and thus reduce the effects of imaging noise on cocaine-induced euphoria and craving. Eleven right-handed men (34±7 years old) with the DSM-IV diagnosis of cocaine dependence were studied. During imaging, subjects underwent a randomized double-blind infusion of either cocaine HCl (0.6 mg/kg up to maximum dose of 40 mg) or saline. Subjects were scanned on an Instascan device (1.5 T General Electric Signa; modified by Advanced NMR Systems, Wilmington, Mass.) using a head coil (General Electric). Six experimental slices were placed along the oblique axial plane, covering the brainstem from medulla to inferior colliculus. Automated shimming with second order shims was performed to improve Bo field homogeneity. Functional scans utilized cardiac gating with an asymmetric spin echo, T2*-weighted CVA (TR=6 RR, three slices per each of the first two RRs; TE=70; offset=−25 ms; 128×64 matrix; thickness=3.12×3.12 mm, through-plane resolution=7 mm; 200 images/slice). Comparison of results without vs. with T1-correction showed no gain from the T1-correction. Seven matched sets of cocaine and saline infusion scans were interpretable after motion-correction. Statistical maps were constructed using Kolmogorov-Smirnov (KS) statistics and thresholded at the corrected p value for brain regions sampled, p<10-5. Anatomic localization of activations was performed using previously defined conventions. Compared to the pre-infusion baseline, cocaine produced focal positive signal changes (FIG. 13) in 5 or more of the 7 subjects in the right NAc, amygdala, and mPFC. Bilateral activation in the VT was also noted. The regions that responded to cocaine showed no activation in response to saline infusion.

### 7.2: Activation of Reward Circuitry After Low-Dose Morphine in Controls

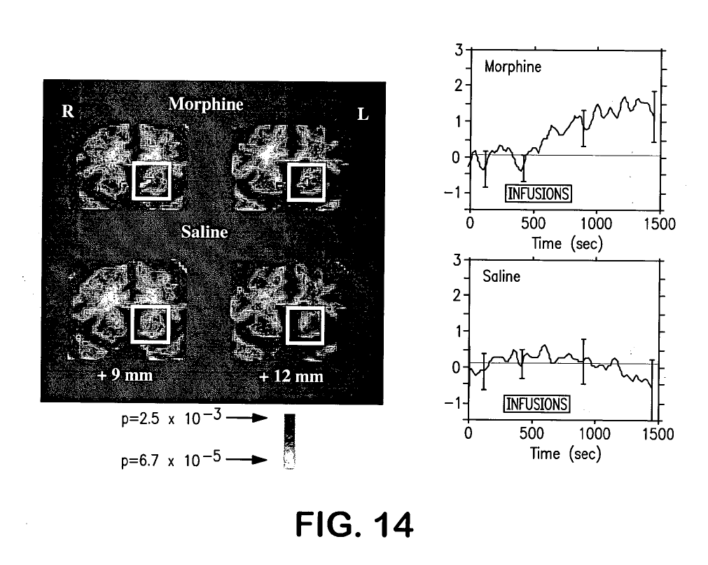

Normal human reward circuitry was studied during low-dose morphine infusions using fMRI with cardiac gating in an unblinded study. Eight right-handed, drug naive, male subjects (age=28±6) were recruited for this experiment, and useful data was obtained in 5 subjects who had no motion artifact. Morphine and saline infusions were counter-balanced across subjects, and segregated in separate scan sessions 7 days apart. Four 2 mL infusions were given over 8 minutes, with each infusion lasting 20 seconds. The onset of each infusion was separated by a 2-minute interval. The total morphine dose divided evenly over the 4 infusions was 4 mg/70 kg. Respiratory rate, heart rate, O₂ saturation, and end-tidal carbon dioxide concentration in the expired air (ETCO₂) were monitored during the experiment in each individual for safety. Subjects were asked to rate their subjective high on a 10-point hedonic rating scale (0=no high; 10=max. high imaginable). No adverse side-effects were noted from morphine; rather, this low dose reliably produced a state of mild euphoria (data not shown), concurrent with significant focal activations in bilateral NAc (FIG. 14) and mPFC along with subthreshold activation in the amygdala, demonstrating that these regions are important to reward functions in the non-addicted human brain. Control conditions with focal brush stimulation and physiology monitoring argue that these results were specific and focal. The observation of left VT activation with cardiac gating implicates dopaminergic systems in the reward circuitry and as a mediator of the subjective responses to other drugs of abuse. The activation of NAc, amygdala and VT by morphine is similar to that seen previously with cocaine, and supports the hypothesis that these regions of reward circuitry serve a generalized function in mediating the rewarding effects of multiple categories of rewarding stimuli.

### 7.3: Acute Cortisol Administration Triggers Craving in Cocaine Dependence

To assess cocaine/stress cross-sensitization, 12 cocaine-dependent individuals (age: 39.8±3.5 years) were administered cortisol (0.2 and 0.5 mg/kg), along with cocaine (0.2 mg/kg) and saline via intravenous boluses, in a double-blind, counterbalanced fashion. The individualized description of subjective responses were categorized into four components: craving, high, rush, and low, which were self-rated on a computerized continuous scale of 0 (none) to 3 (extreme) each minute for two minutes prior and 20 minutes following each infusion. Cocaine craving was proactively defined with the subject, clinically as an urge to use the drug and operationally in terms of the action the individual wanted to engage in to get cocaine. The ratings for high (well-being, self-confidence, and sociability), rush (perception of elevated heart rate and sweating, along with sensations of ‘speeding’), and low (dysphoric affect distinct from the high experience diminishment) were subjectively defined and not necessarily associated with a behavioral response or with the planning of physical activity. ANOVA covaried for both cortisol and craving baseline values revealed significant increases in craving evoked by cortisol, i.e. a time effect (F=3.11; df=20,10; p<0.001), but no dose effect (F=0.06; df=1,10; p=0.81) or dose by time interaction (F=0.50; df=1,10; p=0.97). Cocaine elevated measures of craving (p<0.001); high (p<0.02); rush (p<10-4); and low (p=0.05). Saline produced no significant ratings changes. These results suggest that cocaine dependent subjects may be sensitized to the effects of the stress hormone, cortisol subjectively perceived as craving.

### 7.4: Modulation of α₂-Adrenergic Receptors in PG

Yohimbine is an α₂ receptor antagonist and is an FDA-approved medication (oral formulation) for the treatment of male erectile dysfunction. Its half-life is about 45 minutes (Guthrie et al, 1990) and the peak noradrenergic effect is reached within 10 minutes (Guthrie et al, 1993). Blockade of presynaptic α₂ adrenoceptors results in releases of norepinephrine, both at the central cites and in the periphery leading to subjective stress responses (Kaplan and Sadock, 1998). In addition to its actions on the α₂ adrenergic systems, yohimbine also affects D₂, α₁, 5HT₁ₐ, and benzodiazepine receptors (Egli et al, 2005; Ghitza et al, 2005; Lee et al, 2004). However, blockade of yohimbine's effects by α₂ agonists, clonidine and lofexidine, and replication of these effects by the selective α₂ adrenoceptor antagonist RS-79948-197, renders non-α₂ receptors-related effects unlikely mechanisms of yohimbine's stressogenic action (Ghitza et al, 2005; Le et al, 2005; Lee et al, 2004). Given the central role of nor-adrenaline in the brain sensitization mechanisms (FIG. 1), yohimbine is uniquely suited for the investigation of the gambling/stress cross-sensitization.

To date, numerous clinical studies employed intravenous yohimbine as a pharmacological stressor in neuroimaging and in clinical studies involving healthy subjects and neuropsychiatric patients with depressive, psychotic and anxiety disorders as well as those with SUDs and with post-traumatic stress disorder. A previous ^{‘8}F PET study of ten subjects with PTSD and matched healthy controls during yohimbine infusions (0.4 mg/kg) revealed PTSD-related deactivations in prefrontal, temporal, parietal, and orbitofrontal cortexes that were associated with increased anxiety self-reports. Another neuroimaging study in healthy subjects reported association between cerebral blood flow in the PFC and yohimbine-induced anxiety responses. The dose of yohimbine ranged between 0.125 mg/kg to 0.4 mg/kg and was generally well tolerated. However, the reported adverse effects of yohimbine in clinical trials were as following: elevated blood pressure and heart rate, psychomotor agitation, irritability, tremor, headaches, skin flushing, dizziness, urinary frequency, nausea, vomiting and perspiration.

In this study, subjects diagnosed with PG are administered yohimbine or saline control and perform the monetary reward task as described above. PG subjects administered yohimbine have significantly elevated levels of brain activity in the NAc and other stress-associated regions compared to PG subjects administered saline and normal controls (non-PG subjects).

### 7.5: Reduced NAc Activation in PG Subjects Following Adrenergic Inhibition

In this study, subjects diagnosed with PG are administered an α₂ agonist, a β-adrenergic receptor antagonist, or vehicle control. The PG subjects administered vehicle control demonstrate elevated brain activity, measured by fMRI, in the NAc. By contrast, the PG subjects administered either the α₂ agonist or the β-adrenergic receptor antagonist have significantly reduced NAc activity.

### 7.6: Drug Screening Method for Identifying Compounds Useful for Treating Impulse Control Disorders

The following method may be used to identify candidate compounds capable of treating ICDs. The method is useful for screening any class of compounds (i.e., compounds active at an adrenergic receptor or those with no significant adrenergic activity).

In normal subjects and those with PG, it is expected that yohimbine administration will cause an increase in gambling activity relative to the control state (i.e., no yohimbine). Successful candidate compounds are capable of reversing or eliminating the yohimbine-induced increase in gambling activity. These compounds may be further screened, in the absence of yohimbine, using a larger population of subjects diagnosed as having PG.

Subjects are given an initial cash stipend for participating in the study and are informed that they will be given a choice between earning additional cash and gambling while at the scanner. During an orientation session, subjects complete a brief questionnaire to estimate how much they value gambling. The questionnaire consists of systematic choices of money versus gambling activity (i.e., a value with equal likelihood of gambling or money choice). This personal gambling value is used to set the cost of gambling options during the fMRI imaging sessions.

At the beginning of each imaging block subjects are given 36 tokens, each worth 25% of the subject's personal monetary value, estimated from the questionnaire, for one round on a roulette-type game. During the choice phase, subjects are offered eight optional games that the subjects can “buy” with the tokens, but the tokens left unspent will be redeemed for cash at the end of the session. Four of the gambling options are given a standard price (e.g., four tokens), two gambling options are given an inexpensive price (e.g., two tokens), and two gambling options are given an expensive price (e.g., eight tokens). The cost of the game is varied across the trials in order to: 1) test for potential effects of stress on cost/benefit considerations; 2) test the sensitivity of the procedure i.e., whether choice is related to price and 3) ascertain that decisions are made at the time of the purchase opportunity and are not planned in advance.

Thus, during each trial subjects first sees the “price” of the game (anticipation) and then chooses whether to buy a game or to retain the tokens when the word “Choose” appears above the “price” (Choice). Following the choice, subjects wait for a brief period (Wait), after which the number of tokens and gambling rounds are displayed (Outcome). The Outcome is followed by a display of the total number of tokens and gambling rounds for the whole imaging session (Total) and a visual fixation point (Fixation).

The task lasts for about 45 minutes and consists of 16 imaging blocks with 8 trials. Combining two personal gambling value options (4 tokens) with the two cheap (2 tokens) options and two other personal gambling value options with the two expensive (8 tokens) options results in 8 ordering possibilities. Thus, each sequence will be viewed twice during the 16 blocks.

Subjects are repeatedly tested and are administered either vehicle control, yohimbine, the candidate compound alone, or the candidate compound in combination with yohimbine prior to testing. Individual study designs will vary based on a variety of variables unique to each study including, for example, the number of subjects, number of candidate compounds to be tested, and statistical considerations. Drug administration is performed in a double-blinded manner.

It is expected that subjects diagnosed as suffering from PG opt to will play more of the optional gambling choices and, on average, will risk a greater number of tokens than normal (non-PG) subjects. Yohimbine administration will increase the number of games played and/or the number of tokens risked by both groups of subjects. Useful candidate compounds reduce the number of games played and/or the number of tokens risked by both groups.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method for treating an impulse control disorder in a subject, said treatment comprising administering a therapeutically effective amount of a β adrenergic antagonist, wherein said β adrenergic antagonist is the only neuroactive agent administered to said subject.

2. The method of claim 1, wherein said impulse control disorder is selected from the group consisting of binge eating disorders, intermittent explosive disorder (IED), kleptomania, pathological gambling, pyromania, tricholtillomania, compulsive shopping/buying/spending, repetitive self-mutilation, nonparaphilic sexual addictions, severe nail biting, compulsive skin picking, personality disorders with impulsive features, attention deficit hyperactivity disorder, and substance use/abuse disorders.

3. The method of claim 1, wherein said impulse control disorder is pathological gambling.

4. The method of claim 1, wherein said β adrenergic antagonist inhibits the biological activity of the β1-adrenergic receptor.

5. The method of claim 1, wherein said β adrenergic antagonist inhibits the biological activity of the β2-adrenergic receptor.

6. The method of claim 1, wherein said β-adrenergic antagonist is selected from the group consisting of propranolol, metoprolol, atenolol, nadolol, pindolol, labetalol, acebutolol, timolol, betaxolol, carteolol, carvediol, oxprenolol, nebivolol, sotalol, pronethalol, alprenolol, esmolol, butoxaminer, and ritodrine.

7. The method of claim 1, wherein said impulse control disorder is further treated using a non-pharmacological treatment.

8. The method of claim 7, wherein said non-pharmacological treatment is psychiatric counseling.

9. A method for treating an impulse control disorder in a subject, comprising administering to said subject a therapeutically effective amount of an α2 agonist.

10. The method of claim 9, wherein said impulse control disorder is selected from the group consisting of binge eating disorders, intermittent explosive disorder (IED), kleptomania, pathological gambling, pyromania, tricholtillomania, compulsive shopping/buying/spending, repetitive self-mutilation, nonparaphilic sexual addictions, severe nail biting, compulsive skin picking, personality disorders with impulsive features, attention deficit hyperactivity disorder, and substance use/abuse disorders.

11. The method of claim 9, wherein said impulse control disorder is pathological gambling.

12. The method of claim 9, wherein said α2 agonist is selected from the group consisting of clonidine, guanfacine, lofexidine, methyldopa, guanabenz, tizanidine, and xylazine.

13. The method of claim 9, wherein said method further comprises administering a β-adrenergic antagonist.

14. The method of claim 13, wherein said β-adrenergic antagonist is selected from the group consisting of propranolol, metoprolol, atenolol, nadolol, pindolol, labetalol, acebutolol, timolol, betaxolol, carteolol, carvediol, oxprenolol, nebivolol, sotalol, pronethalol, alprenolol, esmolol, butoxaminer, and ritodrine.

15. The method of claim 13, wherein said α2 agonist and said β-adrenergic antagonist are administered simultaneously.

16. The method of claim 13, wherein said β2 agonist and said β-adrenergic antagonist are administered in the same pharmaceutical formulation.

17. The method of claim 13, wherein said α2 agonist and said β-adrenergic antagonist are administered in different pharmaceutical formulations.

18. The method of claim 9, wherein said subject is administered a non-pharmacological treatment.

19. The method of claim 18, wherein said non-pharmacological treatment is psychiatric counseling.

20. A composition comprising: (i) a β-adrenergic antagonist and (ii) an α2 agonist.

21. The composition of claim 20, wherein said β-adrenergic antagonist is selected from the group consisting of propranolol, metoprolol, atenolol, nadolol, pindolol, labetalol, acebutolol, timolol, betaxolol, carteolol, carvediol, oxprenolol, nebivolol, sotalol, pronethalol, alprenolol, esmolol, butoxaminer, and ritodrine.

22. The composition of claim 20, wherein said α2 agonist is selected from the group consisting of clonidine, guanfacine, lofexidine, methyldopa, guanabenz, tizanidine, and xylazine.

23. The composition of claim 20, wherein said composition is suitable for intravenous, intramuscular, or subcutaneous injection.

24. The composition of claim 20, wherein said composition is suitable for oral administration.

25. The composition of claim 20, wherein said α2 agonist and said β-adrenergic antagonist is present in an amount sufficient to provide therapeutic brain concentrations of each.
